(19) United States
(12) Patent Application Publication
(10) Pub. No.: US 2024/0108835 A1
(43) Pub. Date: Apr. 4, 2024

(54) POSITIVE AIRWAY PRESSURE SYSTEMS AND METHODS FOR TREATING SLEEP DISORDERED BREATHING

(71) Applicant: SleepRes, LLC, Murfreesboro, TN (US)

(72) Inventors: William Noah, Pleasant View, TN (US); Bernard Hete, Kittanning, PA (US); Michael Knepper, Friedens, PA (US)

(21) Appl. No.: 18/536,800

(22) Filed: Dec. 12, 2023

Related U.S. Application Data
(63) Continuation-in-part of application No. PCT/US2022/045897, filed on Oct. 6, 2022.
(60) Provisional application No. 63/253,027, filed on Oct. 6, 2021.
(60) Provisional application No. 63/348,257, filed on Jun. 2, 2022.
(60) Provisional application No. 63/398,775, filed on Aug. 17, 2022.
(60) Provisional application No. 63/462,856, filed on Apr. 28, 2023.
(60) Provisional application No. 63/499,412, filed on May 1, 2023.
(60) Provisional application No. 63/588,467, filed on Oct. 6, 2023.

(51) Int. Cl. A61M 16/00; A61M 16/06

(52) U.S. Cl. CPC A61M 16/022; A61M 16/0003; A61M 16/0616; A61M 2016/0021; A61M 2016/0027; A61M 2016/0042

(57) ABSTRACT

A method for treating sleep disordered breathing includes providing pressure into a patient's airway over a period of time that includes a plurality of inhalation periods and a plurality of exhalation periods. The pressure is at a relief positive airway pressure during a portion of the inhalation periods including an end of the inhalation period, and the pressure is at a therapy positive airway pressure greater than the relief positive airway pressure during a portion of the exhalation periods including an end of the exhalation period. The pressure decreases from the therapy positive airway pressure to the relief positive airway pressure after the end of the exhalation period.

### RELATED APPLICATIONS

This application is a continuation-in-part of International Patent Application No. PCT/US22/45897, filed on Oct. 6, 2022, which claims the benefit of U.S. Provisional Application Ser. No. 63/253,027, filed Oct. 6, 2021, U.S. Provisional Application Ser. No. 63/348,257, filed on Jun. 2, 2022; and U.S. Provisional Application Ser. No. 63/398,775, filed on Aug. 17, 2022, the entire disclosures of which are incorporated herein by this reference. This application further claims priority from U.S. Provisional Application Ser. No. 63/462,856, filed on Apr. 28, 2023; U.S. Provisional Application Ser. No. 63/499,412, filed on May 1, 2023; and U.S. Provisional Application Ser. No. 63/588,467, filed on Oct. 6, 2023, the entire disclosures of which are incorporated herein by this reference.

### TECHNICAL FIELD

The presently-disclosed subject matter relates to positive airway pressure systems and methods for treating sleep disordered breathing such as obstructive sleep apnea. In particular, in the systems and methods of the present invention, the pressure provided into the patient's airway is intentionally reduced from a therapy positive airway pressure to a relief positive airway pressure after the end of the exhalation period and maintained at a lower pressure through the inspiration period and into the next exhalation period.

### BACKGROUND

Sleep apnea is a potentially serious disorder where breathing repeatedly stops and starts during sleep. Some estimates state that over 25 percent of adults between the ages of 30 and 70 years have sleep apnea, with that number rising to as high as 90 percent in some elderly males.

One of the most common treatments for sleep apnea is continuous positive airway pressure (CPAP) therapy. During therapy, a CPAP machine provides air flow through a mask or in-nose nasal pillow to increase air pressure in the patient's throat, which prevents their airway from collapsing. As shown in FIG. 2, a continuous pressure is provided by the CPAP machine during both inhalation and exhalation. CPAP also increases functional residual capacity of the lung, which pulls on and stiffens the pharyngeal airway. The pressure provided during CPAP treatment varies from patient to patient, but the generally held belief is that a minimum pressure is required to keep the patient's airway open during the inhalation period allowing for the flow of air into the patient's respiratory system. Although CPAP therapy can be effective for treating sleep apnea, the air flow and pressure provided through the mask often causes discomfort, which leads to a patient's discontinuing use.

Referring now to FIG. 3, subsequent generations of positive airway pressure therapy were designed to reduce pressure at the beginning of the exhalation period (typically between 5-10 cmH₂O) in an attempt to improve patient comfort. As there are two different pressures provided, these systems are commonly referred to as bi-level positive airway pressure (BPAP) therapy. A driving principle behind BPAP therapy was that it is necessary to maintain the treatment level pressure during inhalation as this is when the airway would collapse. It was commonly held that so long as pressure was maintained during inhalation, equivalent pressure during exhalation was not required for therapy. As such, BPAP machines would reduce pressure during the expiratory phase (EPAP) but maintain inspiratory pressure (IPAP). This thinking led to the belief that maintaining pressure during the inspiratory phase is an imperative. In fact, one commonly held belief is that EPAP “cracks the airway open” and IPAP “blows it open.” Historically, this thinking, which probably emanated from the proliferation of bi-level therapy, has led engineers to believe that IPAP must be maintained at all costs, but EPAP can be sacrificed as needed to improve comfort.

Referring now to FIG. 4, as an expansion of the BPAP system, certain variations arose in which the pressure during exhalation decreases proportionally to the exhalation air flow of the patient rather than a sharp drop at the beginning of expiration. In these systems, the maximum pressure drop is usually less than BPAP and is typically limited to 1-3 cmH₂O. Consistent with the belief that pressure must be maintained during the inspiratory phase, as shown in FIG. 4, the pressure throughout inhalation, including the end of inhalation, is maintained at the maximum required therapeutic level.

Referring now to FIG. 5, as a lower cost alternative to positive airway pressure systems, passive systems were developed in which a check valve allows inspiratory flow to enter with very little resistance, while passing expiratory flow through a fixed pneumatic restriction during exhalation. As such, and as shown in FIG. 5, pressure during inhalation is unaffected by the device and pressure during exhalation increases relative to the exhalation air flow of the patient. To be clear, there is no pressure supplied during inhalation whatsoever, but because of the design of the device itself, pressure begins to increase immediately upon exhalation.

Continued research on the mechanism underlying sleep disordered breathing and methods of treating the same have resulted in a variety of differing and oftentimes contradictory conclusions. While the common understanding in the field continues to emphasize the necessity of higher inspiratory positive airway pressure, in one paper from 2015 entitled “Expiratory and inspiratory positive airway pressures in objective sleep apnea: how much pressure is necessary? A different point of view” published in Volume 2, Issue 6 of the Journal of Lung, Pulmonary & Respiratory Research, the authors suggested the use of physical devices that increase resistance, and thus pressure, during expiration but that had no effect on inspiratory pressure, similar to the passive devices discussed with respect to FIG. 5. As noted in the paper, current BPAP machines do not allow EPAP to be set to zero. As reflected in FIG. 6, the paper also hypothesizes that independent control of the inspiratory and expiratory pressure should not be limited to having IPAP greater than EPAP, but rather should allow IPAP to be set lower than EPAP (as shown with the triangle line) or near zero (as shown with the circle line). Such “inverted” BPAP machines can then be set to work like an EPAP device where pressure changes still occur immediately and are held steady during the respective inhalation and exhalation.

In another paper from 1993 entitle “Dynamic Upper Airway Imaging during Awake Respiration in Normal Subjects and Patients with Sleep Disordered Breathing” published in Volume 148 of the American Review of Respiratory Disease, the authors found with respect to wakeful breathing: (1) the upper airway was significantly smaller in apneic subjects than normal subjects, especially at the retropalatal low and retroglossal anatomic levels; in apneic patients, the airway had an anterior-posterior configuration unlike the normal airway, which had a horizontal configuration with the major axis in the lateral direction; (2) in all three subject groups, little airway narrowing occurred in inspiration, suggesting that the action of the upper airway dilator muscles balanced the effects of negative intraluminal pressure. In apneic patients there was more enlargement of the airway in early inspiration, presumably reflecting increased upper airway muscle dilator activity; (3) in expiration, positive airway pressure resulted in expansion of the airway; this expansion was largest in the apneic patients, indicating that the apneic airway was more distensible than the normal airway; and (4) at the end of expiration, the upper airway narrowed significantly, especially in the apneic patients. While these findings were specific to wakeful breathing, and the authors specifically noted that no conclusions could be drawn to what occurs during sleep or if the subject is breathing through their mouth, the paper nevertheless hypothesized that, should the same results be true during sleep, an increase in pressure near the end of exhalation, as reflected in FIG. 7, may prevent a reduction in airway dimensions. However, it also cautioned that any pressure during early exhalation is not necessary and could be uncomfortable by causing resistance to the patient's exhalation. In the many years since this paper's publication, it is believed that no positive airway pressure system has ever been built that utilizes the concepts proposed in this article.

Despite the wide range of systems and methods researched and developed over many decades, there has continued to be poor adherence by patients to positive airway pressure treatments due to discomfort. A new means of approaching the treatment for sleeping disorders would be both highly desirable and beneficial.

### SUMMARY OF THE INVENTION

The presently-disclosed subject matter relates to positive airway pressure systems and methods for treating sleep disordered breathing such as obstructive sleep apnea. In particular, in the systems and methods of the present invention, the pressure provided into the patient's airway is intentionally reduced from a therapy positive airway pressure to a relief positive airway pressure after the end of the exhalation period and maintained at a lower pressure through the inspiration period and into the next exhalation period.

Unlike traditional systems and methods in which inspiratory positive airway pressure (IPAP) is greater than expiratory positive airway pressure (EPAP), according to the systems and methods of the present invention, pressure is generally lower during the inhalation period as compared to during the exhalation period. It has been found that this increases patient comfort while providing similar treatment efficacy as traditional CPAP or BPAP machines in which IPAP is always greater than or equal to EPAP.

The present invention utilizes the viscoelastic properties of airway tissue by forcing expansion of the tissues later in the exhalation period to maintain the airway during the early portion of the inhalation period until lung volume increases, at which point tracheal traction maintains patency of the airway without any additional pressure support.

When the cross-sectional area of airways is lower, there is an increased resistance and reduced air flow, which increases the likelihood of an obstructive event, such as an apnea. However, by applying pressure to the patient's airways, a force is transmitted to the tissue of the airways where it is stored, or “charged.” It is important to recognize that the amount of “charge” imparted into the tissues is dependent not just on the magnitude of the force, but also the duration during which the force is applied. Specifically, as the tissues have both an elastic and viscous response, a sudden force may result in an elastic response, but this is immediately reversed as soon as the force is removed. That is to say, the tissues elastically return to their original shape immediately after the force is removed. In order to elicit a viscoelastic response where the tissues will remain in the enlarged shape for a period of time, the force must be applied over a period of time. In other words, once the viscoelastic response occurs, after the force is removed, there is a delay in the tissues returning to the narrower shape.

By focusing the pressure application to the end of the exhalation period and into the beginning of the inhalation period, it is possible to have a lower pressure during the remainder of the inhalation period resulting in a reduction of mean airway pressure, and improving overall patient comfort, which is known to improve adherence to therapy.

Due to this new understanding, the pressure profile resulting from the present invention is unlike any previously known positive airway pressure system. Specifically, unlike all previous systems, according to some exemplary implementations of the present invention, the pressure is reduced well before the end of the inhalation period and maintained at a lowered pressure during not only a beginning of the exhalation period but for a period of time thereafter.

According to some exemplary implementations of the present invention, a method for treating sleep disordered breathing includes providing pressure into a patient's airway over a period of time that includes a plurality of inhalation periods and a plurality of exhalation periods. The pressure is at a relief positive airway pressure (RPAP) during a portion of the inhalation periods including an end of the inhalation period. The pressure is at a therapy positive airway pressure (TPAP) during a portion of the exhalation periods including an end of the exhalation period. The TPAP is greater than the RPAP, and the pressure decreases from the TPAP to the RPAP after the end of the exhalation period. The timing in which the pressure transitions between the TPAP and the RPAP is not tied directly to the timing of the transition between the inhalation periods and the exhalation periods. Rather, the pressure is intentionally decreased from the TPAP to the RPAP through a relief transition (RT) that typically occurs during an early portion of the inhalation period. Similarly, the pressure increases from the RPAP to the TPAP through a therapy transition (TT) that typically occurs partway through the exhalation period.

According to some exemplary implementations, the patient chooses the RPAP that is equal to or above a predetermined minimum pressure.

According to some exemplary implementations, the TPAP is maintained for a period of time between about 0 seconds and about 2 seconds after the beginning of the next inhalation period.

According to some exemplary implementations, the TPAP is maintained for a period of time until a triggering event occurs, including, but not limited to: an inhalation volume after the beginning of the next inhalation period reaching an inhalation volume threshold value; an air flow after the beginning of the next inhalation period reaching an air flow threshold value; and zero patient flow.

According to some exemplary implementations, the period of time the TPAP is maintained is proportional to a difference between the RPAP and the TPAP (i.e., a comfort setting), such that, when the difference is greater, the TPAP is maintained for more time before the end of the exhalation period.

According to some exemplary implementations, the period of time the TPAP is maintained is proportional to a difference between the RPAP and the TPAP, such that, when the difference is greater, the TPAP is maintained for more time before the end of the exhalation period.

According to some exemplary implementations, the difference between the RPAP and the TPAP (i.e., a comfort setting) is greater, a period of time the RPAP is maintained during the exhalation period is reduced such that the TPAP is maintained for more time before the end of the exhalation period.

According to some exemplary implementations, the TPAP and the period of time the TPAP is maintained are configured to maintain the patient's airway so as to avoid obstructive events.

According to some exemplary implementations, the RPAP is configured to reduce mean airway pressure and thus improve comfort of the patient.

According to some exemplary implementations, the RPAP is maintained for substantially all of the inhalation period.

According to some exemplary implementations, the RPAP is maintained for a period of time until a triggering event occurs, including, but not limited to: until an air flow after the beginning of the next exhalation period reaches an air flow threshold value; and zero patient flow.

According to some exemplary implementations, the period of time the RPAP is maintained is proportional to a difference between the RPAP and the TPAP, such that, when the difference is greater, the RPAP is maintained for less time before the end of the exhalation period.

According to some exemplary implementations, the pressure increases during the TT period according to a sigmoidal curve. In some exemplary implementations, the pressure increases during the TT period proportionally to at least one of: an air flow in the patient's airway; the TPAP; and the comfort setting.

According to some exemplary implementations, during the inhalation period, the pressure decreases from the TPAP to the RPAP instantaneously. In some other exemplary implementations, the pressure decreases during the RT period according to a sigmoidal function. In some exemplary implementations, the pressure decreases during the RT period proportionally to at least one of: an air flow in the patient's airway; the TPAP; and the comfort setting.

According to some exemplary implementations, during the relief transition period, the pressure provided within the patient's airway decreases from the TPAP an initial amount before maintaining substantially constant at an intermediate pressure and subsequently decreasing in one or more additional steps from the intermediate pressure to the RPAP. According to some specific implementations, the pressure provided within the patient's airway decreases from the intermediate pressure to the RPAP after peak inhalation flow and/or before peak exhalation flow.

An exemplary system for treating sleep disordered breathing includes a flow generator and a conduit operably connected to the flow generator. The conduit has an outlet configured to connect to a patient's respiratory system via a patient interface to provide pressure into a patient's airway over a period of time that includes a plurality of inhalation periods and a plurality of exhalation periods.

According to some exemplary embodiments, the patient interface is a full face mask, a partial face mask, or a nasal pillow, and the RPAP, the TPAP, or both the RPAP and the TPAP are adjusted depending on the type of patient interface connected to the patient's respiratory system.

### BRIEF DESCRIPTION OF THE DRAWINGS

FIG. 1A is a schematic representation of an exemplary positive airway pressure system of the present invention during the inhalation phase of the patient's respiratory cycle;

FIG. 1B is a schematic representation of the exemplary positive airway pressure system of FIG. 1B during a portion of the expiration phase of the patient's respiratory cycle;

FIG. 2 is a graph of a pressure profile for a prior art continuous positive airway pressure system (CPAP) in relation to a graph of a patient's air flow;

FIG. 3 is a graph of a pressure profile for a prior art bi-level positive airway pressure system (BPAP) in relation to a graph of a patient's air flow;

FIG. 4 is a graph of a pressure profile for another prior art positive airway pressure system in relation to a graph of a patient's air flow in which the applied pressure decreases proportionally to the exhalation air flow of the patient;

FIG. 5 is a graph of a pressure profile for a prior art passive airway pressure system in relation to a graph of a patient's air flow;

FIG. 6 is a graph of contemplated pressure profiles in relation to a graph of a patient's air flow theorized by prior art researchers;

FIG. 7 is a graph of another contemplated pressure profile in relation to a graph of a patient's air flow theorized by prior art researchers;

FIG. 8 is a graph of one exemplary applied pressure profile for a positive airway pressure system of the present invention in relation to a graph of a patient's air flow;

FIG. 9 is a graph of another exemplary applied pressure profile for a positive airway pressure system of the present invention in relation to a graph of a patient's air flow; and

FIG. 10 is three comparative graphs of exemplary applied pressure profiles for a positive airway pressure system of the present invention in relation to a graph of a patient's air flow.

### DESCRIPTION OF EXEMPLARY EMBODIMENTS

The presently-disclosed subject matter relates to positive airway pressure systems and methods for treating sleep disordered breathing such as obstructive sleep apnea. In particular, in the systems and methods of the present invention, the pressure provided into the patient's airway is intentionally reduced from a therapy positive airway pressure to a relief positive airway pressure after the end of the exhalation period and maintained at a lower pressure through the inspiration period and into the next exhalation period.

Unlike traditional systems and methods of positive airway pressure treatment in which inspiratory positive airway pressure (IPAP) is greater than or equal to expiratory positive airway pressure (EPAP), according to the systems and methods of the present invention, pressure is generally lower during the inhalation period as compared to during the exhalation period. It has been found that this increases patient comfort while providing similar treatment efficacy as traditional CPAP or BPAP machines in which IPAP is always greater than or equal to EPAP. One method of providing the above benefits previously discovered by the Applicant is the inclusion of a passive resistor provided in the circuit of a positive airway pressure machine, as described in International Patent Application No. PCT/US22/45897, filed on Oct. 6, 2022, which is incorporated herein by reference. However, rather than the inclusion of a passive resistor or other similar device, systems and methods of the present invention actively control the positive pressure delivered to a patient to provide effective pressure therapy while improving patient comfort.

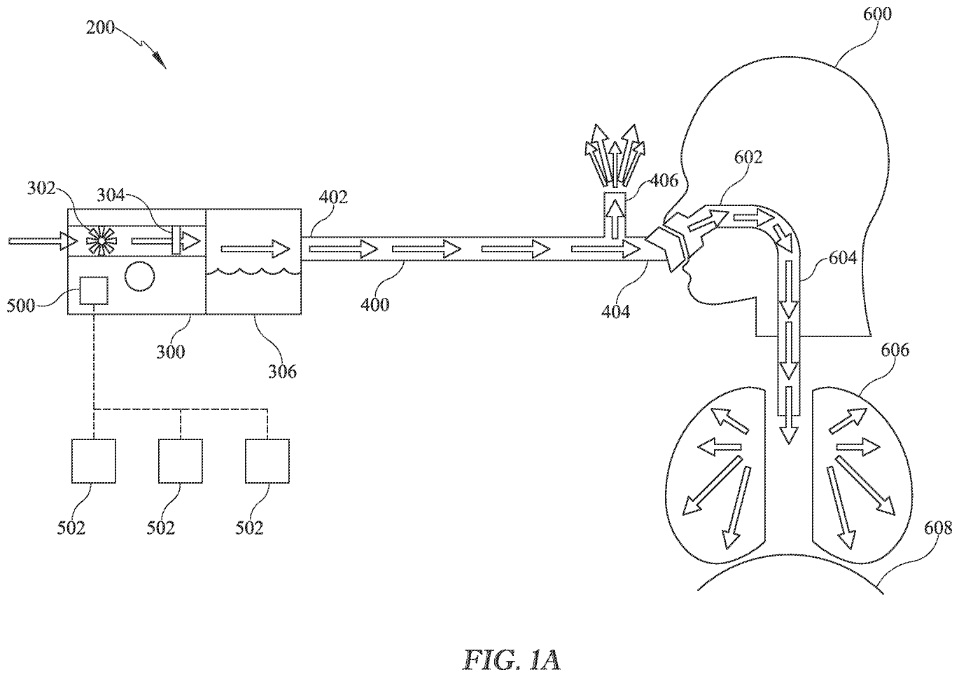

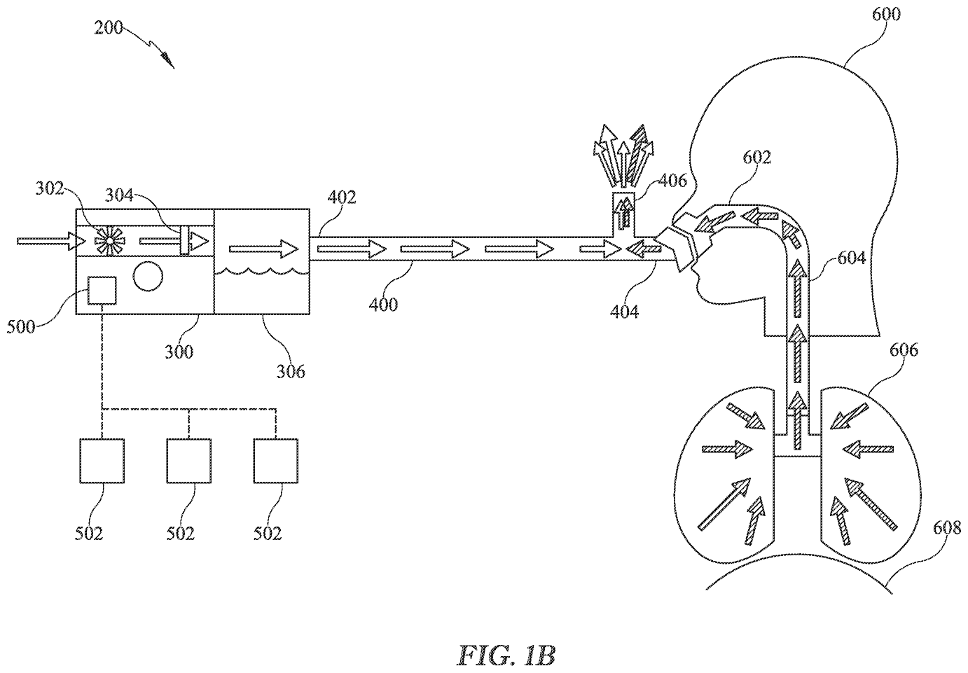

According to some exemplary embodiments, and referring to FIGS. 1A and 1B, a positive airway pressure system 200 of the present invention includes a flow generator 300 to provide a desired air pressure to a patient 600. As such, the flow generator 300 can also be referred to as a pressure generator. Specifically, the exemplary flow generator 300 includes a fan 302 which draws air in from the environment and through a flow meter 304. The air is then directed through a humidifier 306 before passing from the flow generator 300 into a conduit 400 which has an inlet 402 operably connected to the flow generator 300 and an outlet 404 operably connected to the patient's respiratory system as well as an exhaust 406 pathway along the conduit 400 but preferably near the outlet 404. The outlet 404 can be in any suitable form for operably connecting air provided by the flow generator 300 to the patient, such as, but not limited to, a full face mask, a partial face mask, a nasal pillow, or any other suitable outlet. By adjusting the speed at which the fan 302 operates, the flow generator 300 is able to affect the pressure applied by the system 200 to the patient's respiratory system. Others means of adjusting pressure applied to the patient are possible without departing from the spirit and scope of the present invention. For example, another know means of adjusting pressure is an adjustable valve provided on or near the flow generator which bleeds off air flow produced by the fan.

Referring still to FIG. 1A, during an inhalation period of the patient's breathing, the diaphragm 608 drops causing the lungs 606 to expand, drawing air through the nasal passage 602 and pharynx 604. By comparison, and referring to FIG. 1B, during an exhalation period of the patient's breathing, the diaphragm 608 and lungs 606 relax pushing air out through the pharynx 604 and nasal passage 602. Throughout both phases of the patient's respiratory cycle, the flow generator 300 provides pressure through the conduit 400 and the outlet 404 during the inhalation period of the patient's respiratory cycle as well as during the exhalation period of the patient's respiratory cycle. As used herein, the nasal passage 602 is inclusive of the mouth. Also, as used herein, the “airway” of the patient 600 is inclusive of the nasal passage 602, pharynx 604, and lungs 606.

The exemplary airway pressure system 200 further includes a controller 500 for controlling pressure applied to the patient's airway. The controller 500 includes a computer with a processor for executing instructions stored in a memory component to modulate the flow generator 300 and pressure applied to the patient's airway. According to some exemplary embodiments, such active control is obtained via an algorithm that controls the device, for example by adjusting the speed of the fan 302, in accordance with the implementations discussed below. The controller 500 is operably connected to a plurality of sensors 502 that measure different aspects of the system 200, the patient 600, and the ambient environment. The sensors 502 can include, for example, a pressure sensor that monitors the pressure of the air in either the flow generator (e.g., the previously mentioned flow meter 304), the conduit 400, the outlet 404, or the exhaust 406. The sensors 502 can also include a temperature sensor, an ambient pressure sensor, a gauge pressure sensor, a patient flow sensor, ambient humidity sensor, microphone, and accelerometer.

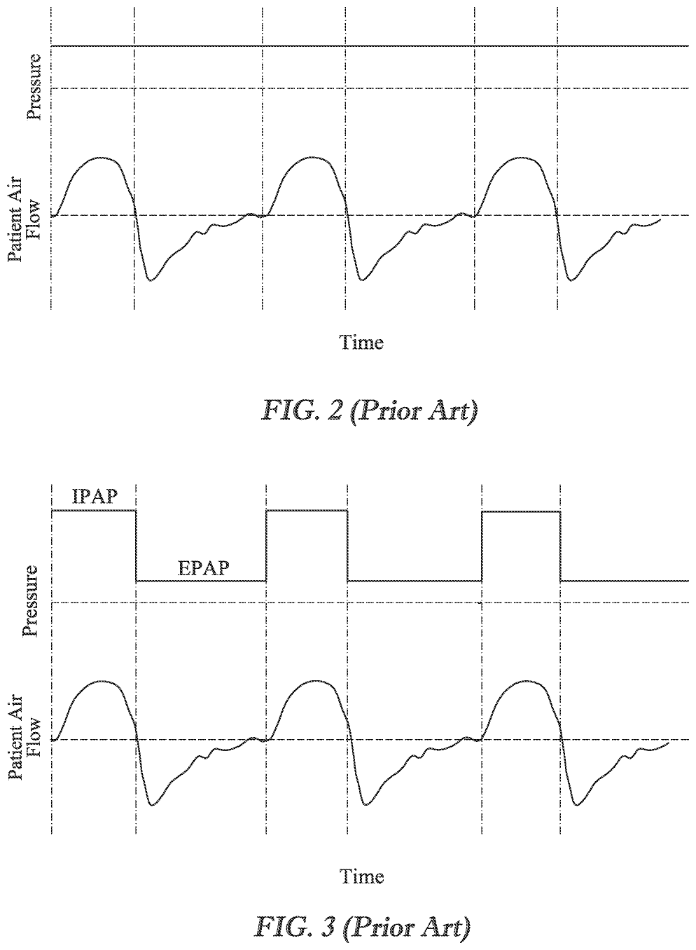

Referring now to FIG. 2, in the most basic applications of positive airway pressure therapy, a continuous pressure is applied during both the inhalation period and the exhalation period, which is commonly referred to as continuous positive airway pressure (CPAP). Referring now to FIG. 3, in other systems, there are two different pressures provided (BPAP). In either case, the pressure provided during treatment varies from patient to patient, but the generally held belief is that a minimum pressure is required to keep the patient's airway open during the inhalation period allowing for the flow of air into the patient's respiratory system.

In known CPAP and BPAP systems, the pressure is maintained for the entirety of the respective inhalation period and exhalation period, even if the pressure changes between these two periods in BPAP. By comparison, in accordance with the present invention, a method of treating sleep disordered breathing focuses on applying a higher pressure primarily, but not exclusively, during a later portion of the exhalation period. The Applicant has discovered that this is the period of time when pressure is most critical for therapeutic effect. As will be discussed in detail below, the present invention utilizes the viscoelastic properties of airway tissue by forcing expansion of the tissues later in the exhalation period. As such, in some instances, the application of the higher pressure extends partially into the subsequent inhalation period to maintain the airway during the early portion of the inhalation period until lung volume increases, at which point tracheal traction maintains patency of the airway. As used herein, “tracheal traction” means the effect of expanding lung volume by the diaphragm moving downward causing the lungs to move downward and thus pulling downward on the trachea, larynx, and pharynx, causing that region to structurally stiffen as a result.

Advantageously, the present invention allows for the pressure to be reduced during the remainder of the inhalation period resulting in a reduction of mean airway pressure and improving overall patient comfort. This is in direct contrast to all current therapy methods in which EPAP is reduced below IPAP in an attempt to improve comfort. Importantly, in the present invention there is still a minimum pressure provided during the entirety of the patient's breath cycle, as discussed further below.

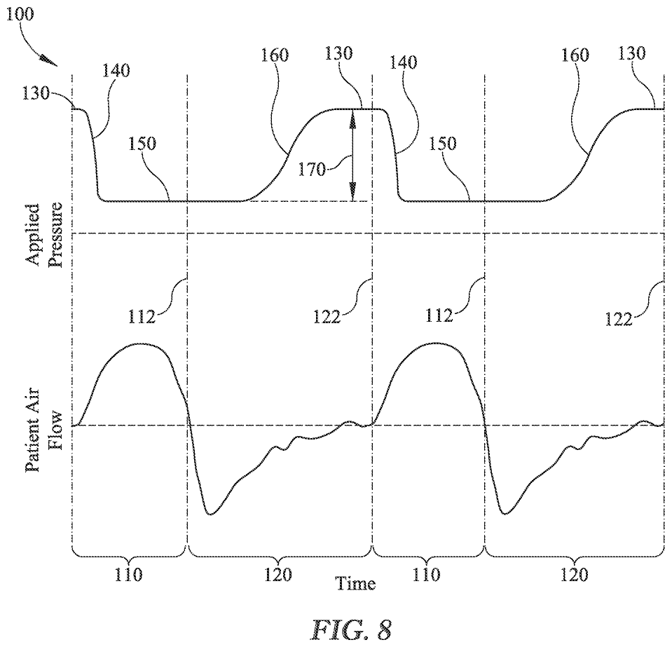

Specifically, and referring now to FIG. 8, in one exemplary implementation of the present invention, throughout a patient's breath cycle, use of the positive airway pressure system 200 of the present invention results in a pressure profile 100 that includes a relief positive airway pressure (RPAP) 150 applied during a portion of the patient's inhalation periods 110 including an end of the patient's inhalation period 112 (i.e., a beginning of the next exhalation period 120) and a therapy positive airway pressure (TPAP) 130 applied during a portion of the exhalation periods 120 including an end of the exhalation period 122 (i.e., a beginning of the next inhalation period 110). Importantly, and unique to the present invention, the TPAP 130 is greater than the RPAP 150. This is to say, in direct comparison to existing BPAP systems where IPAP is greater than EPAP, in accordance with the present invention, on the whole, the pressure provided during the inhalation period 110 is less than the pressure provided during the exhalation period 120 and is typically less than the pressure at the end of exhalation 122.

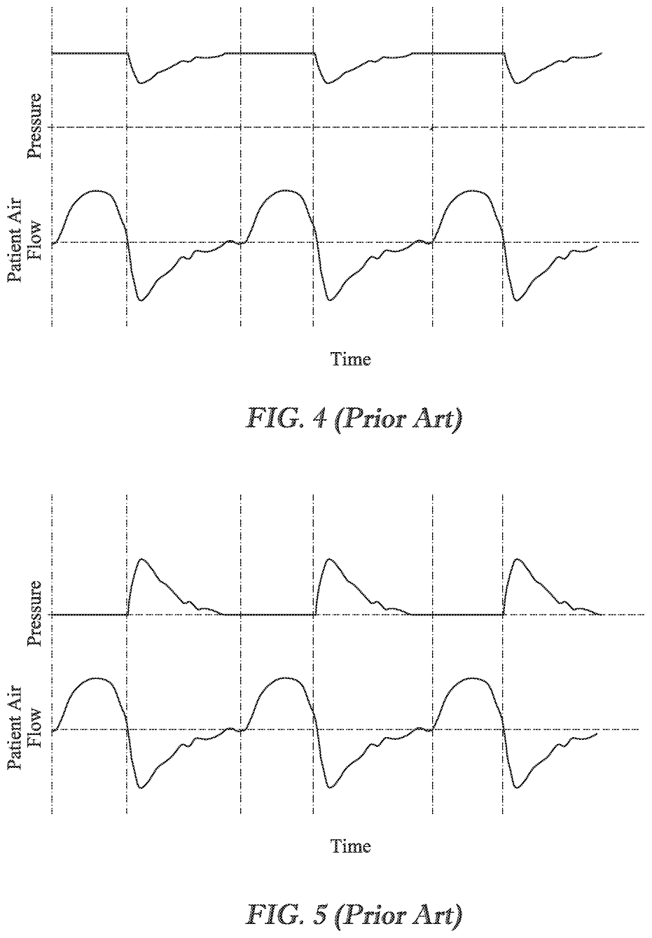

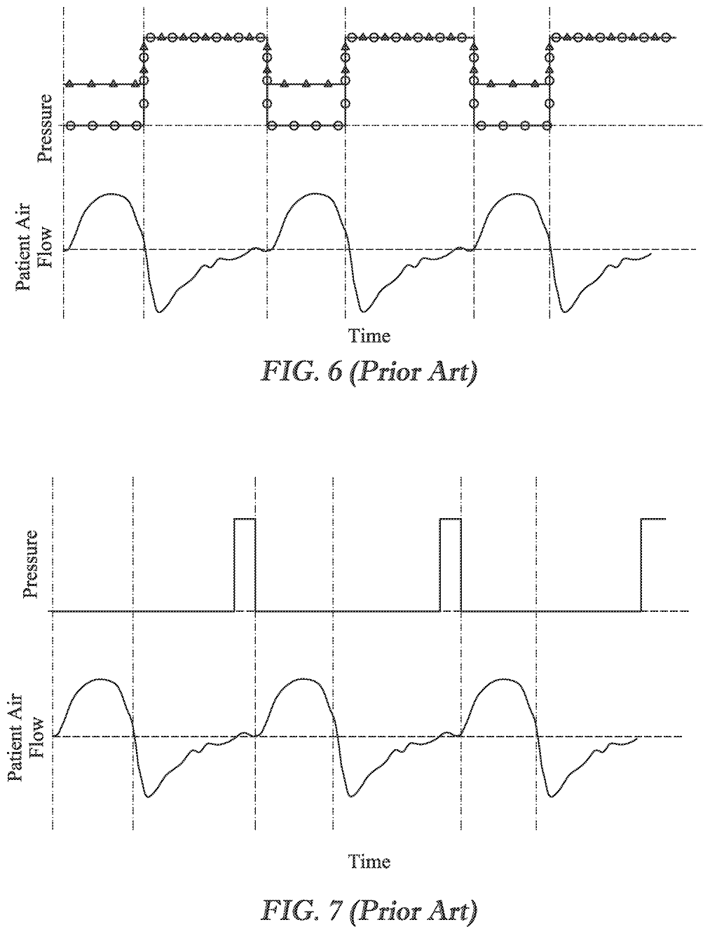

As shown in FIG. 8, the timing in which the pressure transitions between the TPAP 130 and the RPAP 150 is also not tied directly to the timing of the transition between the inhalation periods 110 and the exhalation periods 120. Rather, the pressure is intentionally decreased from the TPAP 130 to the RPAP 150 through a relief transition (RT) 140 that occurs during an early portion of the inhalation period 110. Similarly, the pressure increases from the RPAP 150 to the TPAP 130 through a therapy transition (TT) 160 that occurs partway through the exhalation period 120. Although a certain amount of ramping or transition times are used in some existing machines, before the present invention, the triggering event for these transitions has always been the transition between the inhalation period and the exhalation period and vice versa, as shown for example in FIGS. 3-6. Specifically, prior to the present invention, it was commonly held that IPAP had to be maintained during all, or substantially all of the inhalation period to provide effective therapeutic relief. As discussed in detail below, while the transition between inhalation periods 110 and exhalation periods 120 of the patient's air flow may factor into the onset of the relief transition and/or the therapy transition in some implementations of the present invention, a variety of additional factors are considered in addition to, or instead of, this transition, resulting in a temporal separation of the transition between inhalation periods 110 and exhalation periods 120 of the patient's breathing and the pressure curve caused by the implementation of the present invention.

In the exemplary implementation reflected in FIG. 8, and referring now specifically to the therapy positive airway pressure (TPAP) 130, according to the present invention, the TPAP 130 is configured to maintain the patient's airway so as to avoid obstructive events (e.g., an apnea), as will be described in further detail below. In some exemplary implementations, the TPAP 130 is provided in a range of about 4 cmH₂O to about 25 cmH₂O depending on the particular therapy requirements of a patient. However, TPAP can be provided in a variety of other ranges including, but not limited, to about 5-20 cmH₂O, about 10-15 cmH₂O, and about 8-15 cmH₂O. Although the TPAP 130 shown in FIG. 8 is substantially uniform once the TT 160 is completed, it is contemplated that the TPAP 130 may vary during any given breath cycle between about 0 cmH₂O and about 3 cmH₂O. For example, the TPAP may be based, at least in part, on patient air flow and thus may vary over time as the patient breathes.

As previously mentioned, the TPAP 130 is applied for a period of time sufficiently long enough to maintain the airway during early inspiration until lung volume increases, at which point tracheal traction works to maintain patency of the airway. According to some implementations of the present invention, this period of time is at least 0.5 seconds but the period of time over which the TPAP 130 is maintained may vary between about 0.05 seconds and about 5 seconds depending on a variety of factors discussed further below. According to some exemplary embodiments TPAP is maintained between about 0.5-4 seconds, between about 1-3 seconds, or between about 1-2 seconds.

According to some other implementations, the period of time the TPAP 130 is maintained is determined by one or more environmental parameters including at least one of: atmospheric pressure; ambient temperature; humidity; and elevation above or below sea level. Likewise, according to some other exemplary implementations, the period of time the TPAP 130 is maintained is determined by one or more physiological parameters of the patient including at least one of patient height, patient weight, patient BMI, patient gender, patient age, patient pharyngeal collapsibility, a measured breath rate, a measured I:E ratio, a measured tidal volume, and a measured minute volume. The measured values can be determined based on a single measurement or in some preferred embodiments, based on a plurality of measurements taken over time, such as a rolling average. Variations in clinical requirements for type and duration of CPAP therapy with regard to the above environmental and physiological parameters are well known and readily applied by a person of ordinary skill in the art to the present invention. The effect of the clinical parameters, breath rate, I:E and tidal volume, tend to affect duration directly because of the time of application of TPAP as discussed herein.

In addition to, or instead of, being determined by any of the parameters listed above, in some implementations, the TPAP 130 is maintained until a triggering event occurs. According to some exemplary implementations, the TPAP 130 is maintained until there is substantially zero patient flow, i.e., the beginning of the next inhalation period 110. In some other exemplary implementations, the TPAP 130 is maintained until an inhalation volume after the beginning of the next inhalation period 110 reaches a threshold value (e.g., exceeds the threshold value). In still other exemplary implementations, the TPAP 130 is maintained until an air flow after the beginning of the next inhalation period 110 reaches a threshold value (e.g., exceeds the threshold value). In yet still other exemplary implementations, the TPAP 130 is maintained until a differential (e.g., first, second, or higher order) of air flow after the beginning of the next inhalation period 110 reaches a threshold value. These triggering events are merely exemplary and other triggering events may be used without departing from the spirit and scope of the present invention.

With respect to an inhalation volume threshold in particular, one exemplary inhalation volume that triggers the end of TPAP 130 is 30 mL, but an inhalation volume threshold may include any values in a range of about 10 mL to about 250 mL without departing from the spirit and scope of the present invention. In some implementations, the inhalation volume threshold is determined based on a tidal volume measured during a previous inhalation period 110. For example, in some particular implementations, the inhalation volume threshold is between about 0% and about 50% of a tidal volume measured during a previous inhalation period 110. According to some exemplary embodiments, the inhalation volume threshold is between about 0-40%, about 0-30%, about 0-20%, about 0-10%, about 0-5%, about 1-5%, or about 1-10% of a tidal volume measured during a previous inhalation period. The inhalation volume threshold may also be determined based on an average tidal volume measured during one or more previous inhalation periods 110, for example, at least three previous inhalation periods 110.

With respect to an air flow threshold in particular, one exemplary air flow threshold that triggers the end of the TPAP 130 is 2 L/min, but an air flow threshold may include any value in a range of about 1 L/min to about 10 L/min without departing from the spirit and scope of the present invention. In some implementations, the air flow threshold is determined based on a peak inhalation flow measured during a previous inhalation period 110. For example, in some particular implementations, the air flow threshold is between about 0% and about 50% of a peak inhalation flow measured during a previous inhalation period 110. According to some exemplary embodiments, the air flow threshold is between about 0-40%, about 0-30%, about 0-20%, about 0-10%, about 0-5%, about 0-4%, about 0-3%, about 0-2%, or about 0-1% of a peak inhalation flow measured during a previous inhalation period. The air flow threshold may also be determined based on an average peak inhalation flow measured during one or more previous inhalation periods 110, for example, at least three previous inhalation periods 110.

Of course, it should be readily understood that tidal volume measurements and air flow measurements are typically derived from an estimated patient flow signal, which itself is a filtered version of the machine flow signal. However, other means of directly or indirectly measuring inhalation volume and air flow are possible without departing from the spirit and scope of the present invention.

Furthermore, while a triggering event may immediately cause the end of the TPAP 130 and the beginning of the RT 140, in some implementations, there is additional time after such a triggering event occurs before the end of the TPAP 130 and the beginning of RT 140. For example, according to some exemplary implementations, the TPAP 130 is maintained for between about 0 second and about 2 seconds after a triggering event. In some implementations, the TPAP 130 is maintained for a predetermined period of time after the beginning of the next inhalation period.

In other exemplary implementations, the TPAP 130 is maintained for a variable period of time based on one or more input values. For example, according to some exemplary implementations, the TPAP 130 is maintained for a predetermined period of time after the beginning of the next inhalation period between about 0% and about 50% of an expected length of the next inhalation period 110. In some exemplary implementation, TPAP is maintained for between about 0-40%, about 0-30%, about 0-40%, or about 0-10% of an expected length of the next inhalation period 110. This expected length can be determined by any number of means well known in the art including utilizing a running average of several previous inhalation periods 110 or estimations based in part on metabolic requirements associated with age, gender, and BMI.

Referring now specifically to the relief transition (RT) 140, regardless of how the end of TPAP 130 is determined, the transition from the TPAP 130 to the RPAP 150, i.e., the RT 140, may occur according to a variety of different implementations. As shown in FIG. 8, in one exemplary implementation, during the inhalation period 110, the pressure decreases from the TPAP 130 to the RPAP 150 over a period of time. However, in some other exemplary implementations, the pressure decreases from the TPAP 130 to the RPAP 150 instantaneously. As used herein, “instantaneously” should be understood to mean as soon as practical given the mechanical and physical constraints of the system and human body, which in some embodiments encompasses less than or equal to 1.0 seconds, in some embodiments less than or equal to 0.5 seconds, in some embodiments less than or equal to 0.1 seconds, in some embodiments less than or equal to 0.05 seconds, and in some embodiments less than or equal to 0.01 seconds. According to some implementations, the RT 140 occurs over a period between about 0 seconds and about 2 seconds. However, the period of time is typically not fixed but rather a function dependent upon one or more factors, such as patient flow. Additional information regarding the overall shape of the RT 140 is also discussed further below.

Referring now specifically to the relief positive airway pressure (RPAP) 150, according to the present invention, the RPAP 150 is configured to reduce mean airway pressure and thus improve comfort of the patient, as will be described in further detail below. In some exemplary implementations, the RPAP 150 is provided in a range of about 1 cmH₂O to about 20 cmH₂O. According to some other exemplary embodiments, RPAP is provided in a range of about 1-15 cmH₂O, about 1-10 cmH₂O, or about 1-5 cmH₂O. Regardless, a minimum pressure is maintained throughout the RPAP 150, which is less than the TPAP 130 unless noted otherwise for specific applications and uses. It has been found that maintaining a minimum amount of pressure throughout the entirety of the patient's breath cycle is important for a variety of reasons. Providing zero, or atmospheric, pressure during the inhalation period 110 is not comfortable for a patient because a drop to atmospheric pressure will require greater exertion from the patient's diaphragm to pull a sufficiently negative pressure in the patient's lungs to draw a breath. This is especially uncomfortable if the drop to zero pressure occurs suddenly and near the beginning of inhalation. Furthermore, having no pressure during inspiration will lead to airway narrowing and, as discussed further below, will likely require higher pressure or longer exposure to higher pressure during exhalation. Further still, providing continuous positive pressure maintains fresh air flow through the machine (e.g., the conduit 400 and outlet 404) at all times which helps avoid possible rebreathing of CO₂.

Although the RPAP 150 shown in FIG. 8 is substantially uniform once the RT 140 is completed, it is contemplated that the RPAP 150 may vary during any given breath cycle between about 0 cmH₂O and about 5 cmH₂O. For example, the RPAP 150 may be based, at least in part, on patient air flow and thus may vary over time as the patient breathes. Furthermore, while the RPAP 150 shown in FIG. 8 is the minimum pressure, it is contemplated that the pressure profile may drop below RPAP for a period of time during RT 140, between RT 140 and TT 160, or during TT 160, including pressure temporarily at or near 0 cmH₂O, without departing from the spirit and scope of the present invention. Equally, the pressure may increase above RPAP for a period of time between RT 140 and TT 160 without departing from the spirit and scope of the present invention. Such positive or negative pressure differences occurring between RT 140 and TT 160 can be considered part of the RPAP 150 without departing from the spirit and scope of the present invention.

As previously mentioned, the RPAP 150 is applied for a period of time, but there is no minimum amount of time the RPAP 150 must be maintained. Rather, as discussed below, it is contemplated that the lowered pressure during the RPAP 150 provides improved comfort for the patient while the higher TPAP 130 is more important to the effective treatment of the patient. Accordingly, the time period over which the RPAP 150 is maintained is, in at least some implementations, the result of the time necessary to maintain the TPAP 130 to effectively provide treatment of the patient, as well as the time period required to transition from the TPAP 130 to the RPAP 150 during the RT 140 and to transition from the RPAP 150 to the TPAP 130 during the TT 160. That being said, according to some implementations of the present invention, the period of time that the RPAP 150 is maintained is between about 0.05 and about 5 seconds. As shown in the exemplary implementation of FIG. 8, given the relatively short time period over which the RT 140 occurs, the period of time the RPAP 150 is maintained includes most of the inhalation period 110. Furthermore, it is contemplated that the period of time that the RPAP 150 is maintained extends after the beginning of the next exhalation period between about 0% and about 90% of an expected length of the next exhalation period 120. According to some exemplary embodiments, the period of time that the RPAP is maintained after the beginning of the next exhalation period is between about 10-90%, about 20-90%, about 30-90%, about 40-90%, or about 50-90% of an expected length of the next exhalation period. Although RPAP may typically only extend up to 90% of an expected length of the next exhalation period, according to some exemplary embodiments, the period of time that the RPAP is maintained after the beginning of the next exhalation period is about 91%, about 92%, about 93%, about 94%, about 95%, about 96%, about 97%, about 98%, or about 99% of an expected length of the next exhalation period. In some specific implementations, RPAP extends after the beginning of the next exhalation period 120 for between about 0 seconds and about 5 seconds. According to some exemplary embodiments, RPAP extends after beginning of the next exhalation period for between about 1-4 seconds, about 1-3 seconds, or about 1-2 seconds.

According to some implementations, the period of time the RPAP 150 is maintained is determined by one or more environmental parameters including at least one of: atmospheric pressure; ambient temperature; humility; and elevation above or below sea level. Likewise, according to some other exemplary implementations, the period of time the RPAP 150 is maintained is determined by one or more physiological parameters of the patient including at least one of patient height, patient weight, patient BMI, patient pharyngeal collapsibility, patient gender, patient age, a measured breath rate, a measured I:E ratio, a measured tidal volume, and a measured minute volume. The measured values can be determined based on a single measurement or in some preferred embodiments, based on a plurality of measurements taken over time, such as a rolling average. Once again, variations in clinical requirements for type and duration of CPAP therapy with regard to the above environmental and physiological parameters are well known and readily applied by a person of ordinary skill in the art to the present invention. The effect of the clinical parameters breath rate, I:E and tidal volume, tend to affect duration directly because of the time of application of TPAP as discussed herein. However, RPAP may also be dependent on the feeling of patient comfort while awake.

In addition to, or instead of, being determined by any of the parameters listed above, in some implementations, the RPAP 150 is maintained until a triggering event occurs. According to some exemplary implementations, the RPAP 150 is maintained until there is substantially zero patient flow, i.e., the beginning of the next exhalation period 120. In some other exemplary implementations, the RPAP 150 is maintained until an air flow after the beginning of the next exhalation period 120 reaches a threshold value (e.g., drops below the threshold value after a peak exhalation flow occurs). In yet still other exemplary implementations, the RPAP 150 is maintained until a differential (e.g., first, second, or higher order) of air flow after the beginning of the next exhalation period 120 reaches a threshold value. These triggering events are merely exemplary and other triggering events may be used without departing from the spirit and scope of the present invention.

With respect to an air flow threshold in particular, one exemplary air flow threshold that triggers the end of the RPAP 150 is 2 L/min but an air flow volume threshold may include any values in a range of about 1 L/min to about 5 L/min without departing from the spirit and scope of the present invention. In some implementations, the air flow threshold is determined based on a peak exhalation flow measured during the next exhalation period 120 after RPAP 150 has begun. In some other implementations, the air flow threshold is determined based on a peak exhalation flow measured during a previous exhalation period 120. For example, in some particular implementations, the air flow threshold is between about 10% and about 100% of a peak exhalation flow measured during a previous exhalation period 120. According to some exemplary embodiments, the air flow threshold is between about 10-90%, about 10-80%, about 10-70%, about 10-60%, or about 10-50% of a peak exhalation flow measured during a previous exhalation period 120. Although the air flow threshold is typically at least 10% of the peak exhalation flow, according to some exemplary embodiments, the air flow threshold is about 9%, about 8%, about 7%, about 6%, about 5%, about 4%, about 3%, about 2%, or about 1% of the peak exhalation flow measured during a previous exhalation period 120. The air flow threshold may also be determined based on an average peak exhalation flow measured during one or more previous exhalation periods 120, for example, at least three previous exhalation periods 120. Of course, it should be readily understood that patient air flow is typically estimated by filtering, in some capacity, machine flow, which is measured directly from the system flow meter 304 or other similar sensor. Machine flow is the sum of patient and leak flow, and so filtering can be used to remove the leak flow portion. However, other means of directly or indirectly measuring patient air flow are possible without departing from the spirit and scope of the present invention.

Furthermore, while a triggering event may immediately cause the end of RPAP 150 and the beginning of the TT 160, in some implementations, there is additional time after such a triggering event occurs before the end of the RPAP 150 and the beginning of the TT 160. In some implementations, the RPAP 150 is maintained for a predetermined period of time after the beginning of the next exhalation period. For example, according to some exemplary implementations, the RPAP 150 is maintained for between about 0 seconds and about 2 seconds after a triggering event, e.g., the beginning of the next exhalation period 120. However, such a delay can be applied to any triggering event.

In other exemplary implementations, the RPAP 150 is maintained for a variable period of time based on one or more input values. For example, according to some exemplary implementations, the RPAP 150 is maintained between about 0% and about 80% of an expected length of the next exhalation period 120. According to some exemplary embodiments, RPAP is maintained between about 10-80%, between about 20-80%, between about 30-80%, between about 40-80%, or between about 50-80% of an expected length of the next exhalation period 120. This expected length can be determined by any number of means well known in the art including utilizing a running average of several previous exhalation periods 120 or estimations based in part on metabolic requirements associated with age, gender, and BMI.

Referring now specifically to the therapy transition (TT) 160, regardless of how the end of the RPAP 150 is determined, during the transition from the RPAP 150 to the TPAP 130, i.e., the TT 160, the pressure increases from the RPAP 150 to the TPAP 130 over a period of time. Unlike the RT 140, which can occur quickly, it is considered preferable for the TT 160 to occur over a longer period of time as a sudden increase in pressure would be more readily noticed by the patient and decrease comfort. According to some exemplary implementations, the RT 140 occurs over a relief transition period between about 250 milliseconds and about 5 seconds. However, the period of time is typically not fixed but rather a function dependent upon one or more factors, such as patient flow. Additional information regarding the overall shape of the RT 140 is provided below.

Referring now to both the transitions that occur during the RT 140 and the TT 160, as previously mentioned, the RT 140 and/or the TT 160 may occur over a period of time. In the exemplary implementation shown in FIG. 8, the RT 140 and the TT 160 both provide for a smooth transition between the TPAP 130 and the RPAP 150. However, the shape of the pressure profile 100 during the RT 140 and the TT 160 is not limited and can follow a function, such as an exponential function, a power law function, a parabolic function, a sinusoidal function, and a sigmoidal function. Although a smooth transition is considered preferable, in some embodiments, the shape of the pressure profile 100 during the RT 140 and/or the TT 160 can also be linear.

It is believed that, at least in some circumstances, having a smooth curve is advantageous, as keeping the flow signal smooth avoids problems with false triggering and event detection. The goal is to trigger when the patient's breathing requires it, and not because of some errant perturbation caused by control instability of the CPAP. Providing a smooth transition reduces the likelihood of a false trigger, a false event detection, erroneous flow limitation measurement, and the like. False triggers are more likely to lead to patient discomfort due to a mismatch in the applied pressure and the patient's breathing and, as discussed above, increased discomfort will likely lead to a reduction in therapy usage. Because the RT 140 is more typically initiated near the expiration to inspiration transition, providing a smooth transition without false triggers is of particular importance during the RT 140. By comparison, as TT 160 generally occurs somewhere more towards the middle of the expiratory phase, a false trigger is less likely to be noticed by the patient.

According to some exemplary implementations, the pressure profile 100 during the RT 140 and/or the TT 160 may also be determined based on one or more physiological parameters including at least one of: patient height; patient weight; patient BMI; patient pharyngeal collapsibility; patient gender; patient age; a measured breath rate; a measured I:E ratio; a measured tidal volume; a measured minute volume; a measured exhalation pressure produced by the patient's lungs; a measured exhalation flow produced by the patient's lungs; and a measured peak expiratory flow. The measured values can be determined based on a single measurement or in some preferred embodiments, based on a plurality of measurements taken over time, such as a rolling average. In some specific implementations, the pressure profile 100 during the RT 140 decreases proportional to the increase in the patient's inhalation flow while in some other specific implementations, pressure profile 100 during the RT 140 decreases proportional to the decrease in the patient's inhalation flow after from the peak air flow rate. In still other specific implementations, the pressure profile 100 during the TT 160 increases proportional to the fall in the patient's exhalation flow. It has been shown that matching these changes in pressure to the patient's own breathing, not only in timing but in shape, can help mask the pressure changes and improve patient comfort during the transitions. In turn, this allows for greater pressure changes in instances where the patient may require higher TPAP. As used herein, the terms “proportional” and “proportionally” are not limited to mathematically exact relationships and may include approximate, or relatively closely related, increases or decreases. Furthermore, the terms “proportional” and “proportionally” are used to describe both direct proportionality as well as inverse proportionality.

According to some other implementations, the pressure profile 100 during the RT 140 and/or the TT 160 may also be determined in relation to one or more environmental parameters including at least one of: atmospheric pressure; ambient temperature; humidity; and elevation above or below sea level.

According to yet still other implementations, the pressure profile 100 during RT 140 and/or TT 160 may also be proportional to one or more aspects of the machine flow and pressure provided by the positive airway pressure system 200. For example, the change in pressure during RT 140 and/or TT 160 may occur proportionally to the air flow provided into the patient's airway. As discussed above, air flow measurements are typically derived from an estimated patient flow signal, which itself is a filtered version of the machine flow signal.

Likewise, the pressure profile 100 during the RT 140 and/or the TT 160 may also be proportional to the TPAP 130, the RPAP 150, and/or the difference between the TPAP 130 and the RPAP 150, i.e., the comfort setting 170 that is explained further below.

One exemplary equation contemplated for defining the curve during the RT 140 and/or the TT 160 is produced below in which P_{del} is the difference between the RPAP 150 and the TPAP 130 (i.e., the comfort setting 170); z is time; and β is a coefficient that modifies the shape of the sigmoid. The overall shape of the sigmoid is mapped to the time expected for TPAP 130 and similarly for RPAP 150 to deliver the shape of the sigmoid during each respective phase.

$$\\ P ( z ) = {P}_{de 1} [ \frac{1}{1 + {e}^{- β z}} - 1 ]$$

While FIG. 8 shows only two breath cycles of an inhalation period 110 and exhalation period 120, it should be understood that this is merely exemplary and that the method for treating sleep disordered breathing occurs over a period that includes a plurality of inhalation periods and a plurality of exhalation periods. Furthermore, although the plurality of inhalation periods and plurality of exhalation periods are typically consecutive, it is contemplated that the method of the present invention can be used for non-consecutive inhalation and exhalation periods. That is to say, under certain circumstances, no pressure is applied to the patient's airway for a partial breath cycle, a full breath cycle, or multiple breath cycles. In one particular instance discussed below, this is due to the patient waking up. However, this need not necessarily always be the case, and the methods of the present invention can include one or more partial or full breath cycles where no pressure is applied to the patient's airway as part of the regular and intended therapy treatment.

Further still, although the pressure profile 100 shown in FIG. 8 repeats identically between each breath cycle of an inhalation period 110 and exhalation period 120, it is contemplated that in some exemplary implementations, one or more of the TPAP 130, the RT 140, the RPAP 150, and the TT 160 may vary between breath cycles. Some of these potential changes have been discussed above (e.g., values determined on averages of measurements taken from previous cycles), but it should be appreciated that with respect to the TT 160 in particular, in some implementations of the present invention, the rate at which the pressure increases is modified between exhalation periods 120 to ensure the TPAP 130 is reached during the TT 160 before the end of the exhalation period 120. If TPAP 130 is not reached before the end of the exhalation period 120, time constants for the controller 500 of the positive airway system 200 are updated, which send instructions to increase the pressure during the TT 160 more quickly. Conversely, if the pressure reaches TPAP 130 before a targeted time threshold, the time constants are updated to slow down the rate at which the pressure increases during the TT 160. It is believed that a more collapsible airway might require an earlier return to full pressure during the exhalation period 120 as compared to a less collapsible airway. In such circumstances, it is anticipated that an earlier return to TPAP 130 may be required.

Referring now specifically to the comfort setting 170, as previously mentioned, the comfort setting 170 is the difference between RPAP 150 and TPAP 130. According to some exemplary implementations, the comfort setting 170 is between about 0% and about 50% of the TPAP 130. According to some exemplary implementations, the comfort setting is between about 0-40%, between about 0-30%, between about 0-20% or between about 0-10% of the TPAP. In some specific implementations, the comfort setting 170 is between about 0 cmH₂O and about 15 cmH₂O. Typically, although not necessarily true in all instances, when the TPAP 130 is higher, a larger comfort setting 170 is used and when the TPAP 130 is lower, a smaller comfort setting 170 is used. In some specific implementations, the comfort setting 170 is provided in a range of about 3 and 4 cmH₂O; a range of about 2 and 5 cmH₂O; or a range of about 1 and 6 cmH₂O. According to some exemplary implementations, it is contemplated that a patient may choose any comfort setting 170 so long as the resulting RPAP 150 is equal to or greater than a predetermined minimum pressure. In operation, it is contemplated that a doctor or other professional would prescribe a value, or range, of the TPAP and the patient would then choose a comfort setting. The positive airway pressure system 200 would then use these two inputs, along with any number of other parameters discussed above, to develop a pressure profile. Over the course of multiple days, weeks, months, etc., as a patient acclimates to use of the positive airway pressure system 200, the comfort setting may be changed automatically by the system itself, or manually by the patient and/or doctor. Accordingly, while some comfort settings may be used which do not completely stop all sleep obstructive events, these initial comfort settings may nevertheless increase patient adherence and allow for a later reduction in the comfort setting over time that provides more effective treatment. These adjustments could also be made based on sleep state, as discussed further below.

It should be understood that the various implementations and variations discussed above with respect to TPAP, RT, RPAP, and TT can be combined in any number of ways to arrive at a desired pressure profile in accordance with the present invention. To provide one specific example, the pressure profile may be based primarily on the patient flow rate while ensuring that the applied pressure reaches a predetermined maximum during the TPAP and does not go below a minimum value during the RPAP. Specifically, during the inhalation period, the pressure will decrease from the TPAP to the RPAP proportionally to the decrease in patient inhalation from the peak air flow. Once the pressure reaches the minimum value at the RPAP, the pressure will remain substantially constant throughout the RPAP until the patient air flow reaches peak exhalation during the exhalation period. After peak exhalation, the pressure will increase during the TT proportionally to the decrease in patient exhalation from the peak exhalation air flow until it returns to the maximum value at the TPAP.

Regardless of the particular methods used in developing the pressure profile, as previously mentioned, the TPAP 130 is configured to maintain the patient's airway to avoid obstructive events while the RPAP 150 is configured to reduce mean airway pressure and thus improve comfort of the patient. As used herein, an “obstructive event” may include any instance where the soft tissues of the pharyngeal walls collapse inward and partially obstruct (hypopnea) or fully obstruct (apnea) air flow. The TPAP and the period of time the TPAP is maintained are chosen to increase lung volume at the end of each exhalation period, which can increase tracheal traction on the upper airway.

The present invention utilizes the viscoelastic properties of airway tissue by forcing expansion of the tissues later in the exhalation period to maintain the airway during the early portion of the inhalation period until lung volume increases, at which point tracheal traction maintains patency of the airway without any additional pressure support.

When the cross-sectional area of airways is lower, there is an increased resistance and reduced air flow, which increases the likelihood of an obstructive event, such as an apnea. However, by applying pressure to the patient's airways, a force is transmitted to the tissue of the airways where it is stored, or “charged.” It is important to recognize that the amount of “charge” imparted into the tissues is dependent not just on the magnitude of the force, but also the duration during which the force is applied. Specifically, as the tissues have both an elastic and viscous response, a sudden force may result in an elastic response, but this is immediately reversed as soon as the force is removed. That is to say, the tissues elastically return to their original shape immediately after the force is removed. In order to elicit a viscoelastic response, where the tissues will remain in the enlarged shape for a period of time, the force must be applied over a period of time. In other words, once the viscoelastic response occurs, after the force is removed, there is a delay in the tissues returning to the narrower shape.

As such, the therapy positive airway pressure and the period of time the therapy positive airway pressure is maintained are chosen to extend viscoelastic structures in the airway such that the airway cross-sectional area is expanded viscoelastically. More specifically, the therapy positive airway pressure and the period of time the therapy positive airway pressure is maintained are chosen to increase pharyngeal volume and therefore reduce resistance at regions where an obstruction is likely to occur, even for a period of time after the applied pressure is reduced. By focusing the pressure application to the end of the exhalation period and into the beginning of the inhalation period, it is possible to have a lower pressure during the remainder of the inhalation period resulting in a reduction of mean airway pressure, and improving overall patient comfort which is known to improve adherence to therapy.

The pressure profile resulting from the present invention is unlike any previously developed positive airway pressure system. Specifically, unlike all previous systems, some exemplary pressure profiles of the present invention reduce the pressure well before the end of the inhalation period and maintain a lowered pressure during not only a beginning of the exhalation period but for a period of time thereafter.

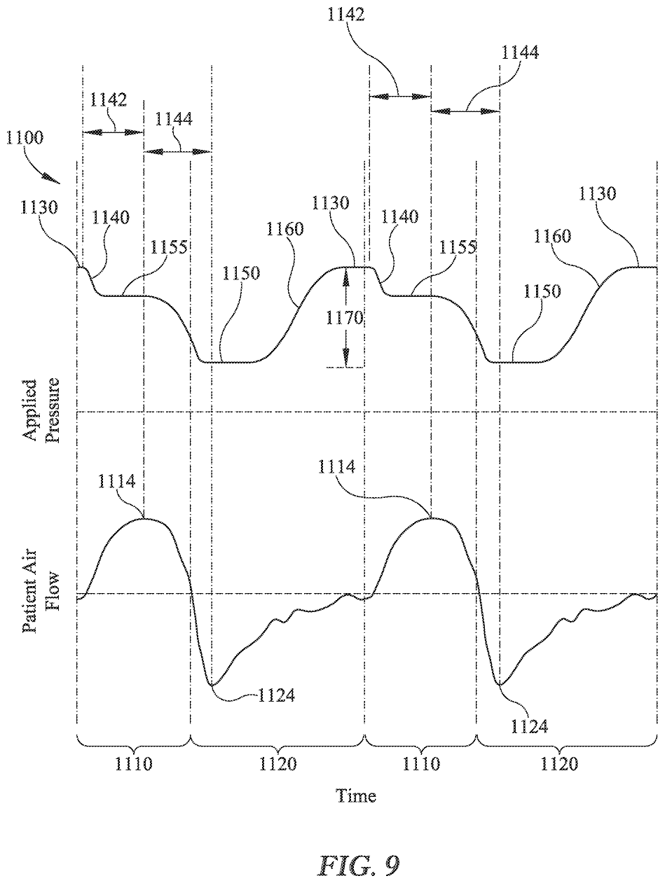

As previously mentioned, the shape of the pressure curve during the relief transition and the therapy transition is not limited. To this end, and referring now to FIG. 9, in some exemplary implementations, the relief transition can occur in a plurality of steps to further enhance comfort. As shown in FIG. 9, and similar to the implementation show in FIG. 8, in the pressure profile 1100 of this exemplary implementation, the pressure decreases from the TPAP 1130 to the RPAP 1150 through a RT 1140 that begins shortly after the beginning of the inhalation period 1110 but still during an early portion of the inhalation period 1110. Similarly, the pressure increases from the RPAP 1150 to the TPAP 1130 through a TT 1160 that occurs partway through the exhalation period 1120. However, unlike the implementation shown in FIG. 8, during the RT 1140 shown in FIG. 9, there is an initial decrease in pressure during a first period of time 1142, which is followed by a subsequent decrease in pressure during a second period of time 1144.

During the first period of time 1142, the pressure provided within the patient's airway decreases from the TPAP 1130 an initial amount before maintaining substantially constant at an intermediate pressure 1155 that is less than TPAP 1130 but greater than RPAP 1150. In this exemplary implementation shown in FIG. 9, the first period of time 1142 ends when the patient's air flow reaches a peak inhalation flow 1114 while the second period of time 1144 ends when the patient's air flow reaches a peak exhalation flow 1124. As such, the overall period of time over which the RT 1140 occurs in FIG. 9 is significantly longer than the RT 140 shown in FIG. 8. However, in other implementations, the first drop in pressure from TPAP 1130 to the intermediate pressure 1155 is done relatively quickly and the second drop in pressure from the intermediate pressure 1155 to RPAP 1150 occurs at or after the peak inhalation flow 1114. To be clear, in these implementations, the second drop begins at or after the peak inhalation flow 1114 and continues to completion at or before the peak exhalation flow 1124. Other timings for the first drop and the second drop are also possible without departing from the spirit and scope of the present invention.

Regardless of the particular timing of the two drops in pressure, it is contemplated that the initial decrease in pressure can be smaller, larger, or substantially equal to the subsequent decrease in pressure. In some exemplary implementations of the present invention, the initial decrease in pressure that occurs during the first time period 1142 is between about 50% and about 80% of the difference between the TPAP 1130 and the RPAP 1150, i.e., the comfort setting 1170. In some other exemplary implementations of the present invention, the initial decrease in pressure that occurs during the first time period 1142 is between about 20% and about 50% of the difference between the TPAP 1130 and the RPAP 1150, i.e., the comfort setting 1170. In some particular implementations, the initial drop in pressure is between about 2 cmH₂O and about 4 cmH₂O. Aside from including two separate pressure drops during the RT 1140, all other aspects of the TPAP 130, RT 140, RPAP 150, and TT 160 described above with respect to FIG. 8 are equally applicable to the TPAP 1130, RT 1140, RPAP 1150, and TT 1160 of FIG. 9. Furthermore, although only two drops in pressure are shown during the RT 1140 in FIG. 9, it is contemplated that three or more drops can occur without departing from the spirit and scope of the present invention.

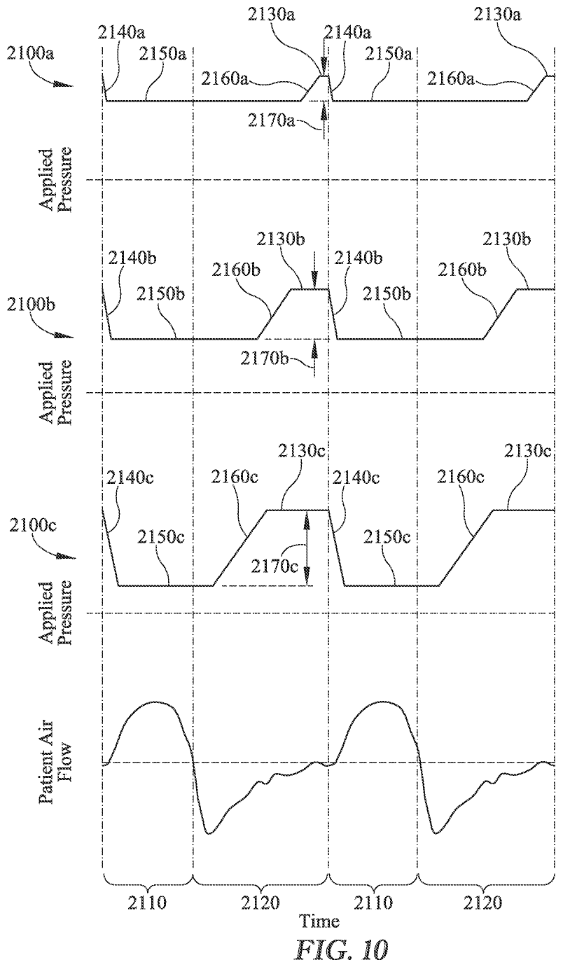

As previously mentioned, the overall shape of the pressure profile of the present invention advantageously takes into account the viscoelastic nature of the patient's airways. Referring now to FIG. 10, three separate exemplary pressure profiles 2100a-2100c are shown, which provide substantially the same therapeutic effect in accordance with the present invention. The pressure profiles shown in FIG. 10 are simplified in some respects as compared to the implementations shown in FIGS. 8-9 and described above. Specifically, and similar to the previously described pressure profiles, in each pressure profile 2100a-2100c shown in FIG. 10 the pressure decreases from the TPAP 2130a-2130c to the RPAP 2150a-2150c through a RT 2140a-2140c and the pressure increases from the RPAP 2150a-2150c to the TPAP 2130a-2130c through a TT 2160a-2160c period. However, unlike the curved RT and TT shown in previous implementations, in each of the pressure profiles 2100a-2100c shown in FIG. 10, the RT 2140a-2140c and TT 2160a-2160c are linear, and the RT 2140a-2140c begins immediately at the beginning of the inhalation period 2110, i.e., the end of the exhalation period 2120.

The second pressure profile 2100b is perhaps most similar to the previous pressure profiles 100, 1100 in that the TT 2160b occurs substantially midway through the exhalation period 2120. As such, the therapeutic effect of the implementation represented by the second pressure profile 2100b is substantially similar to the effects discussed above. Namely, the viscoelastic airway tissues are sufficiently “charged” such that, even after the applied pressure is reduced, the tissues will remain in the enlarged shape for a period of time, thus maintaining the airway during the early portion of the inhalation period until lung volume increases, at which point tracheal traction maintains patency of the airway without any additional pressure support.

As compared to the second pressure profile 2100b, in the first pressure profile 2100a the RPAP 2150a lasts much longer and the TT 2160a does not occur until shortly before the end of the exhalation period 2120. The period of time over which the TPAP 2130a occurs is also shorter compared to the TPAP 2130b in the second pressure profile 2100b. The shorter period in which the pressure is above the RPAP 2150a results in less time for airway tissues to viscoelastically expand from the increased TPAP 2130b. However, the comfort setting 2170a (i.e., the difference between the RPAP 2150a and the TPAP 2130a) of the first pressure profile 2100a is also less than the comfort setting 2170b of the second pressure profile 2100b. In other words, the RPAP 2150a is greater and therefore more pressure is applied to the patient's airways throughout the earlier portion of the exhalation period 2120. As a result, is it unnecessary for TPAP 2130a to be maintained for as long before the end of the exhalation period 2120 in order to provide the same overall effect on the viscoelastic tissues of the patient's airway.

Conversely, in the third pressure profile 2100c, the comfort setting 2170c (i.e., the difference between the RPAP 2150c and the TPAP 2130c) is greater than the comfort setting 2170b of the second pressure profile 2100b, but in the third pressure profile 2100c the RPAP 2150c is shorter, TT 2160c occurs earlier, and the period of length over which TPAP 2130c occurs is longer compared to the second pressure profile 2100b. As such, even with the lower RPAP 2150c, the earlier TT 2160c and longer TTAP 2130c will result in the same overall effect on the viscoelastic tissues of the patient's airway.

As such, while the application of pressure over time is different in each of the pressure profiles 2100a-2100c, the overall therapeutic effect is substantially the same. In other words, the period of time the therapy positive airway pressure is maintained is proportional to a difference between the relief positive airway pressure and the therapy positive airway pressure, such that, when the difference is increased (i.e. the difference is greater), the therapy positive airway pressure is maintained for more time before the end of the exhalation period. Conversely, the period of time the relief positive airway pressure is maintained is proportional to a difference between the relief positive airway pressure and the therapy positive airway pressure, such that, when the difference is increased (i.e., the difference is greater), the relief positive airway pressure is maintained for less time before the end of the exhalation period.

More specifically, when the difference between the relief positive airway pressure and the therapy positive airway pressure is increased, a period of time the relief positive airway pressure is maintained during the exhalation period is reduced such that the therapy positive airway pressure is maintained for more time before the end of the exhalation period

The relationship between the comfort settings 2170a-c and period of time of the corresponding TPAP 2130a-c can be linear, a higher polynomial, power law, exponential, or other common function. Alternatively, or additionally, the relationship could be driven by one or more of the physiological parameters, environmental parameters, or parameters relating to the positive airway pressure system itself (e.g., applied air flow) discussed above. It has been shown through testing that a smooth sigmoid is extremely comfortable, and when implementing the transition, even with a higher difference between RPAP and TPAP, the transition is comfortable and unnoticed when synchronized with patient exhalation. Furthermore, although the pressure profiles 2100a-c are somewhat simplified in FIG. 10, it should be understood that the effect of pressure over time illustrated in FIG. 10 is equally applicable to all of the implementations and variations discussed above with respect to FIGS. 8 and 9 regarding the shape and timing of TPAP, RT, RPAP, and TT.

It should further be appreciated that the methods described above can also vary depending on whether the patient is awake or asleep, or even some state in-between. Generally, comfort has priority while a patient is awake and therapy has priority while a patient is asleep. While the patient is awake, i.e., when comfort is prioritized, it is important to keep the pressure lower during the exhalation period and maintain a lower pressure for as late as possible. However, there still must be an increase at the end of the exhalation period sufficient to maintain airway patency. This can be achieved across a continuum ranging from a high comfort setting for shorter TPAP duration through a small comfort setting but longer TPAP duration. Essentially, a proportional product of these two factors (e.g., comfort setting and time) is preferably maintained for a given patient. Once the patient falls asleep, the degree of comfort required only needs to be maintained such that the patient is not aroused from sleep, so the duration of TPAP can be increased even to the point of eliminating RPAP altogether.

Accordingly, in some exemplary embodiments, the positive airway pressure system 200 is capable of determining whether the patient is awake or asleep and changing one or more parameters of the pressure profile accordingly. For example, the TPAP may be lowered while the patient is awake or in light sleep and increase TPAP gradually as the patient reaches deeper sleep and/or is asleep for longer. Additionally, the timing of TT and/or RT may also be affected based on whether the patient is awake or asleep substantially similar to the pressure profiles discussed above with respect to FIG. 10 except that TPAP is changing rather than RPAP. For example, the positive airway pressure system 200 may initially begin when the patient is awake with a lower TPAP (i.e., a smaller comfort setting) and therefore TT occurs much closer to the end of the exhalation period, similar to the pressure profile 2100a shown in FIG. 10. When the positive airway pressure system 200 determines that the patient is falling asleep, in addition to increasing TPAP, there is a correlated movement of TT away from the end of the exhalation period, similar to the pressure profiles 2100b and 2100c shown in FIG. 10. By beginning TT earlier in the exhalation period, there is more time to gradually transition from RPAP to TPAP while still providing the necessary pressure over time to provide the intended therapeutic effect as discussed above with respect to FIG. 10. Means of determining whether the patient is awake or asleep include, but are not limited to, detecting rapid eye movement (REM); detecting flow limitation or increased upper airway resistance; detecting a response to central sleep apnea; and monitoring respiratory parameter variance (e.g., breath rate, tidal volume, peak flows, etc.) since breathing generally becomes regular once a patient transitions to being asleep.

As previously mentioned, it is contemplated that the above described exemplary implementations may be performed by the positive airway pressure system 200 via an algorithm that controls the device, for example, by adjusting the speed of the fan 302. As a further refinement of the present invention, it is contemplated that one or more physical components could be utilized to create the effects described above. For example, solenoid operated valves can be included in or near the outlet 404 and/or the exhaust 406 of the conduit 400 to selectively raise and lower pressure. Likewise, a fully pneumatic approach utilizing pneumatic function extractors (e.g., a pneumatic square root extractor) may also be used instead of, or in addition to, an electronic control of the fan 302.

Although the above description focuses on the application of the present invention to the treatment of sleep disordered breathing, it should be understood that the system and methods are also applicable for other forms of pressure support, or ventilation therapy, such as mechanical ventilation. Although the specific pressure profiles will vary depending on the type of therapy, many of the concepts regarding the transitions between a therapy positive airway pressure and a relief positive airway pressure are equally applicable as a means to increase comfort during inspiration in substantially the same manner as discussed above. Specifically, while certain ventilation therapy may require the therapy positive airway pressure to extend through more of the inspiration phase, the details regarding the relief transition and therapy transition would still readily apply.

One of ordinary skill in the art will recognize that additional embodiments and implementations are also possible without departing from the teachings of the present invention or the scope of the claims which follow. This detailed description, and particularly the specific details of the exemplary embodiments disclosed herein, is given primarily for clarity of understanding, and no unnecessary limitations are to be understood therefrom, for modifications will become apparent to those skilled in the art upon reading this disclosure and may be made without departing from the spirit or scope of the claimed invention.

What is claimed is:

1. A method for treating sleep disordered breathing, the method comprising:
providing pressure into a patient's airway over a period of time that includes a plurality of inhalation periods and a plurality of exhalation periods;
wherein the pressure is at a relief positive airway pressure during a portion of the inhalation periods including an end of the inhalation period;
wherein the pressure is at a therapy positive airway pressure during a portion of the exhalation periods including an end of the exhalation period, the therapy positive airway pressure greater than the relief positive airway pressure; and
wherein the pressure decreases from the therapy positive airway pressure to the relief positive airway pressure after the end of the exhalation period.

2. The method of claim 1, wherein a difference between the relief positive airway pressure and the therapy positive airway pressure is between about 3 cmH₂O and about 4 cmH₂O.

3. The method of claim 1, wherein a difference between the relief positive airway pressure and the therapy positive airway pressure is less than about 40% of the therapy positive airway pressure.

4. The method of claim 1, further including a step of the patient choosing the relief positive airway pressure that is equal to or above a predetermined minimum pressure.

5. The method of claim 1, wherein the therapy positive airway pressure is maintained for a period of time.

6. The method of claim 5, wherein the period of time extends between about 0 seconds and about 2 seconds after the beginning of the next inhalation period.

7. The method of claim 5, wherein the period of time extends after the beginning of the next inhalation period between about 0% and about 50% of an expected length of the next inhalation period.

8. The method of claim 5, wherein the period of time extends until a triggering event occurs.

9. The method of claim 8, wherein the triggering event is an inhalation volume after the beginning of the next inhalation period reaching an inhalation volume threshold value.

10. The method of claim 9, wherein the inhalation volume threshold value is determined based on a tidal volume measured during one or more previous inhalation periods.

11. The method of claim 8, wherein the triggering event is an air flow after the beginning of the next inhalation period reaching an air flow threshold value.

12. The method of claim 11, wherein the air flow threshold value is determined based on a peak inhalation flow measured during one or more previous inhalation periods.

13. The method of claim 8, wherein the triggering event is zero patient flow.

14. The method of claim 5, wherein the period of time the therapy positive airway pressure is maintained is proportional to a difference between the relief positive airway pressure and the therapy positive airway pressure, such that, when the difference is greater, the therapy positive airway pressure is maintained for more time before the end of the exhalation period.

15. The method of claim 14, wherein, when the difference between the relief positive airway pressure and the therapy positive airway pressure is greater, a period of time the relief positive airway pressure is maintained during the exhalation period is reduced such that the therapy positive airway pressure is maintained for more time before the end of the exhalation period.

16. The method of claim 5, wherein the therapy positive airway pressure and the period of time the therapy positive airway pressure is maintained are configured to maintain the patient's airway so as to avoid obstructive events.

17. The method of claim 1, wherein the relief positive airway pressure is maintained for a period of time.

18. The method of claim 17, wherein the period of time includes substantially all of the inhalation period.

19. The method of claim 17, wherein the period of time extends after the beginning of the next exhalation period between about 0 seconds and about 5 seconds.

20. The method of claim 17, wherein the period of time extends after the beginning of the next exhalation period between about 0% and about 80% of an expected length of the next exhalation period.

21. The method of claim 17, wherein the period of time extends until an air flow after the beginning of the next exhalation period reaches an air flow threshold value.

22. The method of claim 21, wherein the air flow threshold value is a peak exhalation flow during the next exhalation period.

23. The method of claim 21, wherein the air flow threshold value is a predetermined air flow rate less than a peak exhalation flow measured during one or more previous exhalation periods.

24. The method of claim 17, wherein the period of time extends until zero patient flow.

25. The method of claim 17, wherein the period of time the relief positive airway pressure is maintained is proportional to a difference between the relief positive airway pressure and the therapy positive airway pressure, such that, when the difference is greater, the relief positive airway pressure is maintained for less time before the end of the exhalation period.

26. The method of claim 1, wherein the relief positive airway pressure is configured to reduce mean airway pressure and thus improve comfort of the patient.

27. The method of claim 1, wherein, during the exhalation period, the pressure increases from the relief positive airway pressure to the therapy positive airway pressure over a therapy transition period.

28. The method of claim 27, wherein the pressure increases according to a sigmoidal function.

29. The method of claim 27, wherein the pressure increases proportionally to an air flow in the patient's airway.

30. The method of claim 27, wherein the pressure increases proportionally to the therapy positive airway pressure.

31. The method of claim 27, wherein the pressure increases proportionally to a difference between the relief positive airway pressure and the therapy positive airway pressure.

32. The method of claim 1, wherein, during the inhalation period, the pressure decreases from the therapy positive airway pressure to the relief positive airway pressure instantaneously.

33. The method of claim 1, wherein the pressure decreases from the therapy positive airway pressure to the relief positive airway pressure over a relief transition period.

34. The method of claim 33, wherein the pressure decreases according to a sigmoidal function.

35. The method of claim 33, wherein the pressure decreases proportionally to an air flow in the patient's airway.

36. The method of claim 33, wherein the pressure decreases proportionally to the therapy positive airway pressure.

37. The method of claim 33, wherein the pressure decreases proportionally to a difference between the relief positive airway pressure and the therapy positive airway pressure.

38. The method of claim 33, wherein, during the relief transition period, the pressure provided within the patient's airway decreases from the therapy positive airway pressure an initial amount before maintaining substantially constant at an intermediate pressure and subsequently decreasing in one or more additional steps from the intermediate pressure to the relief positive airway pressure.

39. The method of claim 38, wherein the pressure provided within the patient's airway decreases the initial amount of between about 2 cmH₂O and about 4 cmH₂O.

40. The method of claim 38, wherein the pressure provided within the patient's airway decreases the initial amount of between about 20% and about 50% of a difference between the relief positive airway pressure and the therapy positive airway pressure.

41. The method of claim 38, wherein the pressure provided within the patient's airway decreases from the intermediate pressure to the relief positive airway pressure after peak inhalation flow.

42. The method of claim 41, wherein the pressure provided within the patient's airway decreases from the intermediate pressure to the relief positive airway pressure before peak exhalation flow.

43. A method for treating sleep disordered breathing, the method comprising:
providing pressure into a patient's airway over a period of time that includes a plurality of inhalation periods and a plurality of exhalation periods, the pressure varying between a lower relief positive airway pressure and a higher therapy positive airway pressure;
wherein the pressure is at the relief positive airway pressure during a portion of the inhalation periods including an end of the inhalation period and a portion of the exhalation periods including a beginning of the exhalation period.

44. The method of claim 43, wherein the period of time the therapy positive airway pressure is maintained is proportional to a difference between the relief positive airway pressure and the therapy positive airway pressure, such that, when the difference is greater, the therapy positive airway pressure is maintained for more time before the end of the exhalation period.

45. The method of claim 44, wherein, when the difference between the relief positive airway pressure and the therapy positive airway pressure is greater, a period of time the relief positive airway pressure is maintained during the exhalation period is reduced such that the therapy positive airway pressure is maintained for more time before the end of the exhalation period.

46. The method of claim 44, wherein the pressure increases from the relief positive airway pressure to the therapy positive airway pressure at least 0.5 seconds after the beginning of the exhalation period.

47. A method for treating sleep disordered breathing, the method comprising:
providing pressure into a patient's airway over a period of time that includes a plurality of inhalation periods and a plurality of exhalation periods, the pressure varying between a lower relief positive airway pressure and a higher therapy positive airway pressure;
wherein the pressure is at a therapy positive airway pressure during a portion of the exhalation periods including an end of the exhalation period;
wherein, during a portion of the inhalation periods, the pressure is maintained substantially constant at an intermediate pressure less than the therapy positive airway pressure and greater than the relief positive airway pressure; and
wherein the pressure subsequently decreases from the intermediate pressure to the relief positive airway pressure.

48. The method of claim 47, wherein the pressure decreases from the intermediate pressure to the relief positive airway pressure after peak inhalation flow and before peak exhalation flow.

49. A system for treating sleep disordered breathing, the system comprising:
a flow generator; and
a conduit operably connected to the flow generator, the conduit having an outlet configured to connect to a patient's respiratory system via a patient interface to provide pressure into a patient's airway over a period of time that includes a plurality of inhalation periods and a plurality of exhalation periods;
wherein the flow generator provides a relief positive airway pressure during a portion of the inhalation periods including an end of the inhalation period;
wherein the flow generator provides a therapy positive airway pressure during a portion of the exhalation periods including an end of the exhalation period, the therapy positive airway pressure greater than the relief positive airway pressure; and
wherein the pressure decreases from the therapy positive airway pressure to the relief positive airway pressure after the end of the exhalation period.

50. The system of claim 49, wherein the patient interface is a full face mask, a partial face mask, or a nasal pillow; and
wherein the relief positive airway pressure, the therapy positive airway pressure, or both the relief positive airway pressure and the therapy positive airway pressure are adjusted depending on the type of patient interface connected to the patient's respiratory system.

51. The system of claim 49, further comprising a computer with a processor, the processor configured to control the flow generator based on feedback provided from at least one of:
a flow meter configured to measure the air flow provided by the flow generator;
a pressure sensor configured to measure the pressure provided into the patient's airway; and
a patient flow sensor configured to monitor the air flow produced by the patient's lungs.
